(19) United States
(12) Patent Application Publication
(10) Pub. No.: US 2012/0196311 A1
(43) Pub. Date: Aug. 2, 2012

(54) Using cells reprogrammed with oncogenic factors for screening anti-neoplastic agents

(76) Inventor: Shi V. Liu, Apex, NC (US)

(21) Appl. No.: 12/931,439

(22) Filed: Feb. 1, 2011

(51) Int. Cl. C12Q 1/02

(52) U.S. Cl. 435/29

(57) ABSTRACT

Cells reprogrammed with oncogenic factors (CROFs) include incorrectly programmed stem cells such as induced pluripotent stem cells (iPSCs). The present application discloses linkages between iPS reprogramming and its potential roles in neoplastic transformation and thus establishes a foundation for using iPSCs as a class of CROFs for screening anti-neoplastic agents. The screening process involves examining the capability of a single agent or a combination of multiple agents in suppressing a neoplastic process including aerobic glycolysis and the related anabolism and thus inhibiting excessive reproduction of the neoplastic cell. In addition, agents are also screened for their potential in inhibiting invasion and migration of neoplastic cells. Anti-neoplastic agents found through methods disclosed here may represent wide-spectrum anti-neoplastic agents but possess very limited side effect because they target at one or more unique aspects of neoplastic processes, rather than affecting one or more living processes common to all cells including normal cells.

### CROSS-REFERENCE TO RELATED APPLICATIONS

Not Applicable.

### STATEMENT REGARDING FEDERALLY SPONSORED RESEARCH OR DEVELOPMENT

Not Applicable.

### TECHNICAL FIELD

The invention involves methods for screening agents effective in inhibiting neoplastic aerobic glycolysis and anabolism supporting excessive cell reproduction and in reducing invasion and migration of neoplastic cells. More specifically, the invention utilizes cells reprogrammed with oncogenic factors (CROFs) including induced pluripotent stem cells (iPSCs) as means for screening agents that specifically inhibit the neoplastic aerobic glycolysis and anabolism as well as the invasive and metastatic properties of neoplastic cells.

### BACKGROUND OF THE INVENTION

Neoplasia is the abnormal proliferation of cells which means the reproduction of these cells exceeds, and is uncoordinated with, that of the normal tissues around them.

Neoplasm is an abnormal mass of tissue resulted from neoplasia. It may appear as tumor or cancer. Tumor represents a localized swelling formed by a solid neoplasm. Cancer represents a disease in which the neoplastic cells displayed not only uncontrolled growth (reproduction) but also invasion (intrusion on and destruction of adjacent tissues) and sometimes metastasis (dispersion to other locations in the body via lymph or blood). Neoplasm can be benign or malignant. Benign neoplasm often contains highly differentiated cells. Malignant neoplasm such as cancer often contains poorly differentiated or undifferentiated cells.

Despite great progresses in understanding its etiology and pathology neoplasia, especially the malignant neoplasia, still remains as a great risk to human life. This is because there is still a lack of effective treatment for neoplasm, especially the malignant neoplasm. Many conventional anti-neoplastic agents suffer from drawbacks of severe side effects due to their inhibition on some common processes shared by normal cells [1-3]. On the other hand, highly-specific anti-neoplasm drugs that target at the specific “leaf” level genetic mutation unique to different neoplastic cells are not only too narrow spectrum in their anti-neoplastic effect but also suffer from losing effect as surviving neoplastic cells can develop resistance by using alternative routes [4-5]. More ironically, some anti-cancer agents can kill the targeted original cancer cells but meantime lead to the formation of new cancer in the patients [6-8]. The sad reality is that most anticancer drugs [9-10] really do not offer much meaningful benefit to patients' quality of life because they add just an extra week or two suffering time to the patients' lifespan [11-12].

Thus, an urgent need in combating neoplasia is to obtain some “root killers” for neoplastic cells [13]. In other words, we need to find wide-spectrum anti-neoplastic agents that are harmful only to neoplastic cells.

In order to find such a “root” killer of neoplasia, we need to understand the “root” of neoplasia or the “Achilles' heel” of neoplasm.

The “root” of neoplasia or the “Achilles' heel” of neoplasm resides in the unique metabolic properties and the living processes of the neoplastic cells.

A hallmark of all neoplasms is their high rate of glycolysis even under a high oxygen concentration. This phenomenon has been known as Warburg effect since 1930s but remains poorly understood even today [14]. During aerobic glycolysis, pyruvate generated from glucose is not transported into mitochondria for total oxidation for yielding more energy but is converted to lactate in cytosol and then excreted outside the cell [15]. For long time, it is unknown why neoplastic cells would “waste” glucose and choose an energy-“inefficient” metabolism. However, the finding of a “neoplastic or pathological Cori cycle” in which the excreted lactate is carried by the blood to the liver and converted to glucose for reuse by the neoplasm may shed some light in understanding Cachexia, a condition exists in neoplastic patients who suffer massive loss of normal body mass as the neoplasm continues its growth [16]. It turns out that, by avoiding a complete “burn” of glucose to CO₂, neoplastic cells preserved some key carbon “skeleton” for anabolism. The combined result of Warburg effect (the aerobic glycolysis) and pathological Cori cycle (the neoplastic anabolism) thus causes a metabolic imbalance: shifting resource toward neoplastic cells and away from normal cells. This metabolic imbalance ultimately results in systematic failure of patients suffer from a neoplastic disease and their death.

In addition, recent studies have shown that some of the molecular mechanisms underlying the neoplastic metabolism also influence invasion and migration of malignant neoplastic cells which are responsible for metastasis and a wider range of neoplastic diseases [17].

Thus, a very effective and much needed approach for treating neoplasia should be based on inhibiting aerobic glycolysis (Warburg effect), neoplastic anabolism (pathological Cori cycle), or both at the same time. This approach should yield therapeutic schemes that present much less side effects than those associated with conventional anti-cell cycle (reproduction)-based therapy such as chemotherapy or radiotherapy. This approach should also yield much broader spectrum anti-neoplastic drugs than those approaches targeting at some rare mutations specific for only a certain type of neoplastic cells. Understanding unique metabolism common to all or at least most neoplastic cells and finding agents inhibiting such neoplastic metabolism may hit the Achilles' heel of neoplasm and eradicate neoplasm from its root.

In addition to the still lacking full translation of the existing insights into the causes of cancer from bench to bed, a major obstacle in searching for anti-neoplastic agents is ironically the shortage of neoplastic cells suitable for laboratory drug screening [18]. Despite the high incidence of neoplasia, neoplastic cells preserved for research use are relatively few. Collecting primary neoplastic cells is a complex procedure impeded with many red tapes, not to say the great investments and efforts required for characterizing them for research use. Consequently, only a limited number of primary cancer cells have been established as useful cell lines. Thus, there is a need to find more model neoplastic cells for utilization in screening anti-neoplastic agents.

In addition to this limitation in quantity, some neoplastic cell lines passed many times in the laboratories might have accumulated additional mutations that are atypical for natural neoplasm [18]. Thus, it is not uncommon that drugs effective against laboratory lines of cancer cells actually failed dramatically in clinical trails. [19]. Some of the anti-cancer drugs actually lead to opposite effects [20-21]. It is also known that some conventional chemotherapy sometimes induces tumor regression while simultaneously elicits stress responses that protect subsets of tumor cells [22]. Thus, there is a need for finding more appropriate model neoplastic cells suitable for reliable screening of anti-neoplastic agents.

### BRIEF SUMMARY OF THE INVENTION

It is an object of the present invention to identify cells reprogrammed with oncogenic factors (CROFs) as surrogate neoplastic cells for screening anti-neoplastic agents.

It is another object of the present invention to classify induced pluripotent stem cells (iPSCs) as a group of CROFs suitable for screening anti-neoplastic agents.

It is an object of the present invention to provide methods for screening agents, in single or in combination, against neoplastic cells by detecting its or their capability of inhibiting aerobic glycolysis.

It is another object of the present invention to provide methods for screening agents, in single or in combination, against neoplastic cells by detecting its or their capability of inhibiting neoplastic anabolism.

It is an object of the present invention to provide methods for screening agents, in single or in combination, against neoplastic cells by detecting its or their capability of inhibiting invasion, migration and metastasis.

### DETAILED DESCRIPTION OF THE INVENTION

Recently, cells known as induced pluripotent stem cells (iPSCs) have been generated in large quantity and diversity [23-25]. These iPSCs have been described as “indistinguishable” from embryonic stem cells (ESCs) [26-28] and thus been perceived as “ethical” and “safe” replacements of ESCs for cell therapy [29-31] and even regenerative medicine [32-35].

It is a discovery of the present inventor that iPSCs are incorrectly programmed stem cells (still abbreviated as iPSCs) or, in other words, man-made cancer stem cells (mmCSCs) [36-38]. Thus, iPSCs can be classified as a group of CROFs, cells reprogrammed with oncogenic factors, and can be used as replacements for naturally-occurring cancer cells in screening agents against neoplastic cells.

It is a further discovery of the present inventor that iPS reprogramming can be linked with neoplastic aerobic glycolysis and anabolism [39]. Thus, iPSCs can be used as model cells for screening agents that specifically inhibit some aerobic glycolysis and anabolism characteristic for neoplasia.

In addition, it is a discovery of the present inventor that iPSCs may possess invasion and migration capability common to malignant neoplastic cells and thus may also be used for screening agents against the invasion, migration and metastasis of neoplastic cells.

It should be pointed out that the present invention of using iPSCs as a class of CROFs for screening anti-neoplastic agents is fundamentally different from those inventions of using iPSCs as non-cancerous cells for modeling other diseases [40-41] and screening drugs against those diseases [42-43]. As a matter of fact, iPS researchers have been focused on inventing methods for making iPSCs [44] because human iPSCs have been perceived as “less complicated” “human pluripotent cells” than embryonic stem cells (ESCs) and thus are “potentially useful in therapeutic applications in regenerative medicine” [44]. More significantly, claims of generating “cancer-free” “safe” iPSCs suitable for clinical applications are being made repeatedly [28, 45-47], despite the criticisms against the hype contained in these claims [36, 48]. A very recent publication [49] describes acquisition of iPSCs by selecting those cells with transgenes integrated into the so-called “safe harbors”, the genomic regions outside positions known for integration mutation. Even though the iPSCs are still made with the already known oncogenes, a claim of “out of harm's way is still made [50]. This demonstrates the lacking of understanding with regarding to how iPS reprogramming results in neoplastic transformation and thus how similar iPSCs are to cancer cells than to ESCs.

At the present time mainstream iPS researchers and top journals are still rejecting the discovery of iPSCs as man-made cancer stem cells (mmCSCs) [37-38] which identifies some cancer risks for various “cancer-free” iPSCs [23-24, 26, 51-53]. Strong efforts are still being made in promoting iPSCs as “ethical” and “safe” ESC replacements for cell therapy and regenerative medicine [28, 54-58]. A recent publication reporting generating iPSCs from dermal fibroblasts of a patient suffering from Hutchinson-Gilford Progeria syndrome (HGPS) [59] has even been regarded as to “lead to novel insights into mechanisms of aging”[60], even though these HGPS-iPSCs are merely some cancerous cells carrying the mutations for HGPS.

Thus, even though some recent publications have noticed the “similarity” between iPSCs and cancer cells [61-62] or the common path between the generation of iPSCs and CSCs [63], the authors of these publications are still contributing the intriguing “parallel” as a result of partial [28] or incomplete [47] reprogramming. At the end, the intrinsic cancer risk of iPSCs has been neglected even in the “comprehensive review [28] or “straight talk” [47] by the leading iPS researchers. Arguments have also been made that “although there are common pathways activated during reprogramming and tumorigenesis, there are fundamental differences between iPS and transformed cancer stem cells” [63].

However, by clearly identifying the various linkages between iPS reprogramming and neoplastic transformation, it is hoped that the present application will establish a solid foundation for arguing iPSCs as a kind of neoplastic cells very similar to natural cancer cells and thus justifying their use as serendipitous replacements for cancer cells in screening agents against neoplastic cells.

It turns out that Myc (c-Myc), a very important iPS reprogramming factor, is a notorious oncogene and a master transcription factor that integrates cell proliferation with metabolism through its regulation of thousands of genes including microRNAs (miRNA) [64]. In addition to its known function in regulating the cell cycle and glucose metabolism [65], Myc also stimulates glutamine catabolism [66] through the repression of miRNAs miR-23a and miR-23b [67].

More significantly c-Myc enhances the expression of poly-pyrimidine tract binding protein PTB (also known as hnENPI), hnRNA1 and hnRNA2, and leads to selective expression of pyruvate kinase isoform 2 (PKM2) [65]. PKM2 is the M2 splice isoform of pyruvate kinase (PK) [68] which is a key enzyme for aerobic glycolysis [69], as compared with the M1 splice isoform of pyruvate kinase (PKM1) which is a key enzyme for oxidative phosphorylation. Thus the selective expression of specific isoform of PK serves as a toggle switch for shifting mass-energy metabolism between an energy production-efficient oxidative phosphorylation and a mass production-efficient aerobic glycolysis.

It is interesting to notice that PKM2 is the dominant form of PK in embryonic cells and PKM1 is the dominant form of PK in the adult cells [68-69]. This age-specific expression of different isoforms of the same enzyme reflects the physiological need as PKM2 is needed for glycolytic anabolism supporting mass increase in the growth stage of the life and PKM1 is needed for allowing established cells to perform more energy-consuming functions at the grown up stage of the life. Thus a change in the expression of different isoforms of the same enzyme leads to different modes of metabolism in different life stages. Overgrowth of cell mass such as neoplasm is not a desired shift.

Unfortunately, the re-expression of PKM2 [70] activates those “resting” cells and drives them from normally a “quiescent” state into a hyper-proliferation state [71]. This adulthood expression of an embryonic enzyme isoform does not lead to the “rejuvenation” of the whole organism but a formation of some harmful and even deadly neoplasm.

More than just contributing to the transformation of normal cells into neoplastic cells, c-Myc coordinately regulates the expression of 13 different “poor-outcome” cancer signatures [17]. In addition, functional inactivation of MYC in human breast cancer cells specifically inhibits distance metastasis in vivo and invasive behavior in vitro [17]. So c-Myc may also contribute to the acquisition of metastatic capability of the neoplastic cells.

Therefore, iPSCs generated with inducing factors including c-Myc may naturally possess some basic neoplastic features known to natural cancer cells.

The iPSCs generated without c-Myc may also possess oncogenic nature. For example, Lin28, an inducing factor used in place of c-Myc [25], has recently been found in association with cancers [72-74]. More importantly, Lin28 has been shown as a Myc-downstream factor exerting the similar effect as Myc [74-75].

As a matter of fact, iPS reprogramming factors currently employed are more or less associated with various cancers [37-38, 76-77]. Thus, the oncogenic potential is intrinsic for iPS reprogramming, at least for the proto-type iPS reprogramming methods [39]. This intrinsic oncogenic potential is intensified when a tumor-suppressing mechanism is inhibited or knocked out [39]. Unfortunately, many iPS researchers just do not want to face with this dark side of iPS reprogramming and continue at looking at the “bright” side of their discoveries [78]. They emphasize the enhanced “efficiency” of iPS reprogramming and elevated yield of the iPSCs by knocking out the tumor-suppressing mechanisms [79-83] while ignore the increased risks of cancer potential from these tumor suppression mechanism-jeopardized iPSCs [84-85].

Nevertheless, increasing reports are presenting observations of chromosomal aberrations [86] and cancer-related epigenome changes [87] in iPSCs. There are also reports documenting formation of rhabdomyosarcomas iPSCs [88]. These observations have led to some concerns over the “variation” in the safety of iPSCs [89]. But claims of generating “transformation-deficient” [45] and thus “safe-induced pluripotent stem cells (safe-iPSCs) with therapeutic potential” [46] are still being made. It has been believed that “although there are common pathways activated during reprogramming and tumorigenesis, pluripotent stem cells and tumorigenic cells have important differences” and thus “the critical distinctions between true cancer cells and reprogrammed somatic cells may be that reprogrammed cells remain genetically intact” [61]. Thus, despite a clear message arguing the intrinsic distinctions between iPSCs and ESCs [90-92] and some later experimental reports supporting this argument [93-94], leading iPS researchers still reject the intrinsic distinctions between iPSCs and ESCs and the high similarity between iPSCs and cancer cells [95]. The most recent review on iPSCs still claims: “Numerous studies indicate that, at least for some clones, iPSCs are similar if not indistinguishable from ESCs derived from embryo or nuclear transfer experiments” and “somatic cells can be reprogrammed to a pluripotent state, which is molecularly and biologically indistinguishable from that of ESCs” [28] and a straight talk states that there's no reason . . . to think that true bona fide iPSCs cannot function as well as ESCs [47]. This attitude is also reflected by the lack of appreciation of the cancerous nature of iPSCs even by the well experienced stem cell researchers.

Amazingly, even some cancer researchers apparently still lack an understanding of the cancerous nature of the iPSCs. A recent study has found “a Myc network accounts for similarities between embryonic stem and cancer cell transcription programs” [96]. Even though some iPSCs were also included in this study, the report failed in identifying iPSCs as cancer cells. As a matter of fact, the corresponding author of this report even did not answer the straight question from the present inventor on whether or not iPSCs are cancer cells.

However, it is hoped that, continued dissection of the iPS reprogramming process may lead to not only a comprehensive identification of a sufficient factor set for complete and safe somatic to pluripotent reprogramming [97] but also a increased awareness of the neoplastic nature of iPS reprogramming [39, 98]. More importantly, if the application of this invention is placed into practice, the cancerous nature of iPSCs may be made very obvious, if anti-neoplastic agents discovered via methods disclosed in this invention are also very effective in killing natural cancer cells.

It is important to point out that iPS reprogramming can turn normal cells neoplastic even without any genetic modification and a cell reproduction event. This feature will be a key point for the present invention which is focused on discovering anti-neoplastic agents that are effective in inhibiting the metabolic mutation serving as a root process for supporting the malicious competition of neoplastic cells against normal cells.

In the past, cancer research has been heavily focused on genetic mutations, including mutations in mitochondria, as causes for neoplasia [99-100]. The discovery of Warburg effect even led some researchers to believe that neoplastic cells have abnormal mitochondria. The outcome of this genetic cancer dogma is the focus of searching anti-cancer drugs that fix the genetic mutations, including mitochondria mutations [101]. However, many drugs targeting the effects of genetic mutations often fail in killing tumor cells and even succeed in killing normal cells [102].

It turns out that, many times, it is the mitochondrial uncoupling, the abrogation of ATP synthesis by mitochondria, promotes the Warburg effect in some neoplastic cells and contributes to their resistance to chemotherapy targeting mitochondria [103]. These cancer cells may shift to the oxidation of non-glucose carbon sources to maintain mitochondrial integrity and function [103]. More importantly, increased level of c-Myc in cancer cells causes an increase in level of glutaminase, a protein that helps cells convert amino acid glutamine into an energy source. The breakdown of glutamine provides cancer cells a carbon source. In fact, glutamine can serve as a major nutrient for cancer cells [104], especially when facing glucose deprivation [105]. Also worth of notice is that mutation in some genes such as KRAS or BRAF often lead to up-regulation of the expression of GLUT1 (encoding glucose transporter-1) and SGLT1 [106]. Thus neoplastic cells often have enhanced glucose uptake and glycolysis, and can survive even at low glucose concentration [107]. Thus, neoplastic cells may still have normal mitochondria despite their abnormal use. Amazingly, drugs targeting mitochondria sometimes kill normal cells more effectively but exert less or even no harm to neoplastic cells which use less or even shut down their mitochondria.

A metabolic mutation or a metabolism switch may be a predominant feature in cancer cell formation. This change may happen at the epigenetic levels. A simple RNA splicing which is a modification of an RNA transcript through removing of introns and joining of exons may produce different proteins out of the same gene [108-109]. This epigenetic regulation plays a very important role in normal development as well as neoplastic tumorigenesis [110-111]. Very often, the alternative splicing changes the mode of mass/energy metabolism [112-113] and this alteration in splicing can be influenced by the conditions in which the cells reside [114-115]. A very recent study just confirmed that some cancer-related epigenome changes have been found in iPSCs [87].

Studies have shown that oncogenes such as Myc [116-117] play very important roles in contributing to glycolytic metabolism in cancer cells [70]. Studies also show that glucose deprivation induces oncogenic mutations [107]. The fact that hypoxia enhances the generation of iPSCs [118] indicates that iPSCs may have switched into a neoplastic glycolysis.

Thus, it is reasonable to expect that iPSCs can be reliably used as a class of CROFs for screening anti-neoplastic agents that are effective in inhibiting metabolic mutation which serves as a root for neoplastic transformation of normal cells into tumor/cancer cells.

Of course, this proposal is apparently against the current mainstream thinking which regards iPSCs as ethical replacements for ESCs and “cancer-free” and thus even “safe” for cell therapy and regenerative medicine. However, it is right from those reports making “cancer-free” claims for iPSCs that the inventor of this patent application found evidence of the cancer risk for iPSCs [48]. With more detailed reasoning disclosed here for linking iPS reprogramming with neoplastic transformation, iPS researchers should come to a reality that their iPSCs may find a better utility: serving as serendipitous cancer cells for screening anti-neoplastic agents.

According to the present invention, potential therapeutic agents may be screened for their ability for inhibiting aerobic glycolysis in neoplastic cells. Aerobic glycolysis has been studied for many decades now and the technical practitioners in the related research field should be familiar with the arts of how to examine the effect of various agents on the different aspects of aerobic glycolysis. Studies can be also designed to study the specific gene expression and/or the enzyme activity related to an aspect of aerobic glycolysis. There have been many publications reporting such studies and thus methodologies can be easily obtained and followed. But the Seahorse Bioscience approach (http://seahorsebio.com) may provide a very convenient way to achieve these goals. The approach comprises simultaneous measurement of oxygen consumption rate (OCR) and extracellular acidification rate (ECAR) in the presence or absence of the testing compounds and thus determination of the rate of oxidative phosphorylation (OXPHOS) and glycolysis, respectively. A change from OXPHOS to glycolysis is usually the earliest change in the process of oncogenic transformation. On the contrary, an inhibition of glycolysis can be used as an early indicator for the anti-neoplastic potential of the testing agent.

According to the present invention, potential therapeutic agents may also be screened for their ability in reducing the competitiveness of neoplastic cells in neoplastic anabolism. Neoplastic anabolism includes two major aspects: the competitive strength of neoplastic cells in grabbing and utilizing resources for cell mass production and the existence of a pathogenic Cori cycle that provides resources to neoplasm. Any means known in the art to measure reduction in the competitiveness of neoplastic cells and blockage of the pathogenic Cori cycle can be used for checking the effect of the screened agents in affecting these processes in CROFs such as iPSCs. For example, studies can be designed to measure the gene expression or the enzyme activity related to an aspect of neoplastic anabolism.

According to the present invention, potential therapeutic agents may be screened for their ability of inhibiting invasion, migration and metastasis of neoplastic cells. This kind of studies has been routinely performed with other cancer cells and the methods available in published literature can be easily found and followed.

According to the present invention, potential therapeutic agents may also be screened for their ability for inducing death of neoplastic cells. There are many methods for assessing cell viability and some of them are routinely used and thus well known.

It should be pointed out that, despite the nonobvious nature of the present invention, the enablement of this novel invention is not difficult at all. Prior arts in studying effects of agents on cancer cell metabolism, growth, reproduction, invention, and migration are abundant and very easy to find. Nevertheless, some essential enablement descriptions of the present invention are still presented here so that researchers with ordinary skills in the field can carry out the invention without undue experimentation.

One embodiment of carrying out the present invention of screening anti-neoplastic agents using CROFs including iPSCs comprises the following steps:

First, the metabolic status of CROFs such as iPSCs should be evaluated so that their suitability for screening different kinds of anti-neoplastic agents can be assessed and a background control can be established. This metabolism diagnosis can be done with a Seahorse XF extracellular flux analyzer and there are rich information on Seahorse Bioscience website (http://seahorsebio.com/products/xf-analyzers/index.php) for understanding the principles underlying the assays and learning how to perform the various tests. Successful applications revealed in scholar publications can also be found there. If the analysis shows a predominance of glycolysis in the tested cells, then not only the nature of testing cells as bona fide neoplastic cells is confirmed, but also the suitability for working as a model for screening anti-glycolysis broad spectrum anti-neoplastic agents is established.

Secondly, the testing agents will be evaluated on their effects on the metabolism of the CROFs, such as iPSCs. Again, this evaluation can be performed using the Seahorse XF extracellular flux analyzer. If the testing agents can inhibit the glycolysis more than other aspect of the metabolism, then the chance of them serving as effective metabolic root-level anti-neoplastic agents is great and their side-effect may be minimal.

Thirdly, the metabolic effective testing agents can be evaluated further for their capability to kill the testing cells. It is well known in the art that viability of a cell can be determined by contacting the cell with a dye and viewing it under a microscope. The most common dye used in the art for this purpose is trypan blue. Viability of cells can also be determined by detecting DNA synthesis. Cells can be cultured in cell medium with labeled nucleotides, e.g., ³H thymidine. The uptake or incorporation of the labeled nucleotides indicates DNA synthesis. In addition, colonies formed by cells cultured in medium indicate cell growth and is another way to test viability of the cells.

Fourthly and as a particularly suited approach to reduce to practice the present invention, novel anti-neoplastic agent(s) could be tested against CROFs in tetrazolium salt-based metabolic assays such as the XTT assay. As such, the respective CROFs that are maintained and propagated in cell culture medium (e.g. RPMI 1640/10% FCS) could be plated in 96-well plates and then incubated for a given time period (e.g. 48 to 72 hours) with the respective testing agent(s), using no testing agent(s) group as a control. At the end of such incubation, XTT substance (and its activation reagent) would be added and the level of soluble formazan product derived from XTT by cellular enzymes would be measured. The more cell proliferation coinciding with metabolic activity is present, the more formazan would be produced by the cells from XTT. If, however, there is an active anti-neoplastic agent equally present, such formazan production would be significantly reduced as a result of the growth-inhibitory effect of such compound.

All of these mentioned and many other unmentioned cell viability/proliferation tests are well-established and thus do not need any more detailed description here.

Fifthly, the metabolic effective testing agents can be evaluated further for their capability to inhibit the invasion and migration of CROFs including iPSCs. These evaluations can be performed with arts known in the field of cancer research. For example, the invasion and migration capability can be studied using a microfluidic device [119]. The effect on metastasis can be evaluated by established methods.

As a sixth point, the validity of the various anti-neoplastic effects of the selected anti-plastic agents from testing with CROFs such as iPSCs may be confirmed with natural cancer cells that are well characterized. Although this additional test is not an intrinsic component of the present invention it is nevertheless helpful for establishing the present invention as a trustable and reliable method for screening anti-neoplastic agents.

Finally, CROFs could be injected in vivo into nude mice, subsequently observed as to whether they grow out to develop macroscopic tumors, and, if so, treated by means of novel anti-neoplastic drug candidates with primary tumor shrinkage as one of the possible endpoints for drug efficacy.

Further than providing the above guidelines and continuing on illuminating the likely neoplastic processes in CROFs such as iPSCs and thus showcase some potential applications of using CROFs including iPSCs for exploring a variety of anti-neoplastic agents, some additional examples of recent discoveries on metabolic mutation-based neoplastic transformation are presented below. Methods of discovering anti-neoplastic agents described in these reports may also serve as illustrations of additional embodiments of the current invention.

It has been shown that hypoxia and oncogenic mutations drive glycolysis, with the pyruvate to lactate conversion being promoted by increased expression of lactate dehydrogenase A (LDH-A) and inactivation of pyruvate dehydrogenase. The NAD+ pool is consecutively regenerated and supports the high glycolytic flux required to produce anabolic intermediates. Glutaminolysis provides metabolic intermediates such as alpha-ketoglutarate to feed and thereby maintain the tricarboxylic acid cycle as a biosynthetic hub. Glycolysis and glutaminolysis share the capacity to generate NADPH, from the pentose phosphate pathway and through the malate conversion into pyruvate, respectively. Both pathways ultimately lead to the secretion of lactate. More than a waste product, lactate was recently identified as a major energy fuel in tumors. Lactate produced by hypoxic tumor cells may indeed diffuse and be taken up by oxygenated tumor cells. Preferential utilization of lactate for oxidative metabolism spares glucose which may in turn reach hypoxic tumor cells. Monocarboxylate transporter 1 regulates the entry of lactate into oxidative tumor cells. Its inhibition favors the switch from lactate-fuelled respiration to glycolysis and consecutively kills hypoxic tumor cells from glucose starvation. Combination with radiotherapy renders remaining cells more sensitive to irradiation, emphasizing how interference with tumor cell metabolism may complement current anticancer modalities [120]. On the other hand, increased expression and activity of LDH-A were detected in Taxol-resistant cells which showed a higher sensitivity to the specific LDH inhibitor, oxamate. Treating Taxol-resistant cells with the combination of Taxol and oxamate showed a synergistical inhibitory effect on these cancer cells by promoting apoptosis in these cells [121].

Neoplastic cells often possess different capacities than normal cells in dealing with some metabolite [106]. To eliminate lactate and to prevent cellular acidification tumor cells show up-regulation of MCT4, an H+-coupled lactate transporter. In addition, the Na+-coupled lactate transporter SMCT1 is silenced in cancer cells. SMCT1 also transports butyrate and pyruvate, which are inhibitors of histone deacetylases. The silencing of SMCT1 occurs in cancers of a variety of tissues. Re-expression of SMCT1 in cancer cell lines leads to growth arrest and apoptosis in the presence of butyrate or pyruvate, suggesting that the transporter may function as a tumor suppressor. Tumor cells meet their amino acid demands by inducing xCT/4F2hc, LAT1/4F2hc, ASCT2, and ATB0,+. xCT/4F2hc is related primarily to glutathione status, protection against oxidative stress, and cell cycle progression, whereas the other three transporters are related to amino acid nutrition. Pharmacologic blockade of LAT1/4F2hc, xCT/4F2hc, or ATB0,+ leads to inhibition of cancer cell growth.

An epigenetic mechanism of the Warburg effect has been proposed [122]. Fructose-1,6-bisphosphatase-1 (FBP1), which functions to antagonize glycolysis was down-regulated through NF-kappaB pathway in Ras-transformed NIH3T3 cells. Restoration of FBP1 expression suppressed anchorage-independent growth, indicating the relevance of FBP1 down-regulation in carcinogenesis. Indeed, FBP1 was down-regulated in gastric carcinomas and gastric cancer cell lines. Restoration of FBP1 expression reduced growth and glycolysis in gastric cancer cells. Moreover, FBP1 down-regulation was reversed by pharmacological demethylation. Its promoter was hypermethylated in gastric cancer cell lines and gastric carcinomas. Inhibition of NF-kappaB restored FBP1 expression, partially through demethylation of FBP1 promoter.

Using iPSCs as a group of CROFs for screening anti-neoplastic agents/drugs has many advantages which include but are not limited to:

First, there are many different types of iPSCs which are readily available for research use;

Secondly, many iPSCs have been well characterized in their genetic aspects and even some epigenetic aspects and thus further exploration on their metabolic mutation-based neoplastic changes would be more productive than testing on other less known cells.

Thirdly, iPSCs have become a part of some researchers' scientific life. These dedicated iPS researchers seating on the iPS bandwagon would become valuable human resource in carrying out the present invention of using iPSCs as target cells for screening anti-neoplastic agents, if the financial incentive is given for killing (neoplastic) iPSCs rather than creating (therapeutic) iPSCs.

Therefore, once iPS researchers realize that enhancing the efficiency of generating iPSCs is not a boosting of the immortalization but actually an intensification of the neoplastic transformation, then these iPS researchers may be the first followers in chasing the discovery of cancerous iPSCs and become the main force using established iPSCs as surrogates for natural cancer cells to search for broad-cancer-spectrum anti-neoplastic agents. This trend is not obvious at the present time. But with the disclosure of this patent application to the public in the future, this trend will for sure to come.

Thus, with an totally unexpected and even unwelcomed discovery of iPSCs as man-made cancer cells and with a detailed presentation linking iPS reprogramming with neoplastic transformation, it is anticipated that future research on neoplasia, which include all kinds of tumors and cancers, will move into a new horizon. With the finding of anti-cancer drugs that are toxic only to neoplastic but not normal cells, clinical treatment of neoplastic diseases may yield an unprecedented good outcome.

It should be pointed out that the above guidelines and some examples represent just some possible applications and embodiments of the present invention. It should not be understood as limitation and boundary of the present invention. The real scope and the right of intellectual property protection should be based on the claims granted for this patent application.

### REFERENCES

1. Sudakin, V. and T. J. Yen, Targeting mitosis for anti-cancer therapy. BioDrugs, 2007. 21(4): p. 225-33.
2. Gupta, R. and R. M. Brosh, Jr., Helicases as prospective targets for anti-cancer therapy. Anticancer Agents Med Chem, 2008. 8(4): p. 390-401.
3. Florea, A. M. and D. Busselberg, Anti-cancer drugs interfere with intracellular calcium signaling. Neurotoxicology, 2009. 30(5): p. 803-10.
4. Levin, L. A. and H. V. Danesh-Meyer, Lost in translation: bumps in the road between bench and bedside. JAMA, 2010. 303(15): p. 1533-4.
5. Knight, Z. A., H. Lin, and K. M. Shokat, Targeting the cancer kinome through polypharmacology. Nat Rev Cancer, 2010. 10(2): p. 130-7.
6. Cichowski, K. and P. A. Janne, Drug discovery: inhibitors that activate. Nature, 2010. 464(7287): p. 358-9.
7. Hatzivassiliou, G., et al., RAF inhibitors prime wild-type RAF to activate the MAPK pathway and enhance growth. Nature, 2010. 464(7287): p. 431-5.
8. Poulikakos, P. I., et al., RAF inhibitors transactivate RAF dimers and ERK signalling in cells with wild-type BRAF. Nature, 2010. 464(7287): p. 427-30.
9. Van Cutsem, E., et al., Open-label phase III trial of panitumumab plus best supportive care compared with best supportive care alone in patients with chemotherapy-refractory metastatic colorectal cancer. J Clin Oncol, 2007. 25(13): p. 1658-64.
10. Moore, M. J., et al., Erlotinib plus gemcitabine compared with gemcitabine alone in patients with advanced pancreatic cancer: a phase III trial of the National Cancer Institute of Canada Clinical Trials Group. J Clin Oncol, 2007. 25(15): p. 1960-6.
11. Hoag, H., Cancer drugs should add months, not weeks, say experts. Nat Med, 2011. 17(1): p. 7.
12. Ocana, A. and I. F. Tannock, When are “positive” clinical trials in oncology truly positive? J Natl Cancer Inst, 2011. 103(1): p. 16-20.
13. Thompson, C. B., Attacking cancer at its root. Cell, 2009. 138(6): p. 1051-4.
14. Vander Heiden, M. G., L. C. Cantley, and C. B. Thompson, Understanding the Warburg effect: the metabolic requirements of cell proliferation. Science, 2009. 324(5930): p. 1029-33.
15. Hsu, P. P. and D. M. Sabatini, Cancer cell metabolism: Warburg and beyond. Cell, 2008. 134(5): p. 703-7.
16. Tisdale, M. J., Cachexia in cancer patients. Nat Rev Cancer, 2002. 2(11): p. 862-71.
17. Wolfer, A., et al., MYC regulation of a “poor-prognosis” metastatic cancer cell state. Proc Natl Acad Sci USA, 2010. 107(8): p. 3698-703.
18. Borrell, B., How accurate are cancer cell lines?Nature, 2010. 463(7283): p. 858.
19. Emmenegger, U. and R. S. Kerbel, Cancer: Chemotherapy counteracted. Nature, 2010. 468(7324): p. 637-8.
20. Labi, V., et al., Apoptosis of leukocytes triggered by acute DNA damage promotes lymphoma formation. Genes Dev, 2010. 24(15): p. 1602-7.
21. Michalak, E. M., et al., Apoptosis-promoted tumorigenesis: gamma-irradiation-induced thymic lymphomagenesis requires Puma-driven leukocyte death. Genes Dev, 2010. 24(15): p. 1608-13.
22. Gilbert, L. A. and M. T. Hemann, DNA damage-mediated induction of a chemoresistant niche. Cell, 2010. 143(3): p. 355-66.
23. Takahashi, K., et al., Induction of pluripotent stem cells from adult human fibroblasts by defined factors. Cell, 2007. 131(5): p. 861-72.
24. Takahashi, K. and S. Yamanaka, Induction of pluripotent stem cells from mouse embryonic and adult fibroblast cultures by defined factors. Cell, 2006. 126(4): p. 663-76.
25. Yu, J., et al., Induced pluripotent stem cell lines derived from human somatic cells. Science, 2007. 318(5858): p. 1917-20.
26. Okita, K., T. Ichisaka, and S. Yamanaka, Generation of germline-competent induced pluripotent stem cells. Nature, 2007. 448(7151): p. 313-7.
27. Park, I. H., et al., Reprogramming of human somatic cells to pluripotency with defined factors. Nature, 2008. 451(7175): p. 141-6.
28. Hanna, J. H., K. Saha, and R. Jaenisch, Pluripotency and cellular reprogramming: facts, hypotheses, unresolved issues. Cell, 2010. 143(4): p. 508-25.
29. Park, I. H., et al., Disease-specific induced pluripotent stem cells. Cell, 2008. 134(5): p. 877-86.
30. Hanna, J., et al., Treatment of sickle cell anemia mouse model with iPS cells generated from autologous skin. Science, 2007. 318(5858): p. 1920-3.
31. Kiskinis, E. and K. Eggan, Progress toward the clinical application of patient-specific pluripotent stem cells. J Clin Invest, 2010. 120(1): p. 51-9.
32. Salewski, R. P., E. Eftekharpour, and M. G. Fehlings, Are induced pluripotent stem cells the future of cell-based regenerative therapies for spinal cord injury? J Cell Physiol, 2010. 222(3): p. 515-21.
33. Andersson, E. R. and U. Lendahl, Regenerative medicine: a 2009 overview. J Intern Med, 2009. 266(4): p. 303-10.
34. Lee, H., et al., Induced pluripotent stem cells in regenerative medicine: an argument for continued research on human embryonic stem cells. Regen Med, 2009. 4(5): p. 759-69.
35. Amabile, G. and A. Meissner, Induced pluripotent stem cells: current progress and potential for regenerative medicine. Trends Mol Med, 2009. 15(2): p. 59-68.
36. Liu, S. V, iPS cells: a more critical review. Stem Cells Dev, 2008. 17(3): p. 391-7.
37. Liu, S. V., IPS cells are man-made cancer cells. Logical Biology, 2008. 8: p. 16-18.
38. Liu, S. V., Further evidence that iPS cells are man-made cancer cells. Logical Biology, 2008. 8: p. 66-68.
39. Zhang, W. K., et al., Cancer Cell Formation by iPS Techniques: Mechanisms and Testing Approaches Logical Biology, 2009. 9: p. 22-41.
40. Maehr, R., et al., Generation of pluripotent stem cells from patients with type 1 diabetes. Proc Natl Acad Sci USA, 2009. 106(37): p. 15768-73.
41. Dimos, J. T., et al., Induced pluripotent stem cells generated from patients with ALS can be differentiated into motor neurons. Science, 2008. 321(5893): p. 1218-21.
42. Webb, S., iPS cell technology gains momentum in drug discovery. Nat Rev Drug Discov, 2009. 8(4): p. 263-4.
43. Gunaseeli, I., et al., Induced pluripotent stem cells as a model for accelerated patient- and disease-specific drug discovery. Curr Med Chem, 2010. 17(8): p. 759-66.
44. Georgieva, B. P. and J. M. Love, Human induced pluripotent stem cells: a review of the US patent landscape. Regen Med, 2010. 5(4): p. 581-91.
45. Nakagawa, M., et al., Promotion of direct reprogramming by transformation-deficient Myc. Proc Natl Acad Sci USA, 2010. 107(32): p. 14152-7.
46. Tsuji, O., et al., Therapeutic potential of appropriately evaluated safe-induced pluripotent stem cells for spinal cord injury. Proc Natl Acad Sci USA, 2010. 107(28): p. 12704-9.
47. Daley, G., Straight talk with . . . George Daley. Interview by Elie Dolgin. Nat. Med, 2010. 16(6): p. 624.
48. Liu, S. V., Towards a balanced view on iPS Cells. Logical Biology, 2008. 8: p. 32-38.
49. Papapetrou, E. P., et al., Genomic safe harbors permit high beta-globin transgene expression in thalassemia induced pluripotent stem cells. Nat Biotechnol, 2011. 29(1): p. 73-8.
50. Williams, D. A. and A. J. Thrasher, Out of harm's way. Nat Biotechnol, 2011. 29(1): p. 41-2.
51. Aoi, T., et al., Generation of pluripotent stem cells from adult mouse liver and stomach cells. Science, 2008. 321(5889): p. 699-702.
52. Wernig, M., et al., In vitro reprogramming of fibroblasts into a pluripotent ES-cell-like state. Nature, 2007. 448(7151): p. 318-24.
53. Nakagawa, M., et al., Generation of induced pluripotent stem cells without Myc from mouse and human fibroblasts. Nat Biotechnol, 2008. 26(1): p. 101-6.
54. Vogel, G., Breakthrough of the year. Reprogramming Cells. Science, 2008. 322(5909): p. 1766-7.
55. Vogel, G., Cellular reprogramming. New technique RIPS open stem cell field. Science, 2010. 330(6001): p. 162.
56. Editors of Science, The Runners-up: Souped-up cellular reprogramming. Science, 2010. 330: p. 1605.
57. Yamanaka, S, and H. M. Blau, Nuclear reprogramming to a pluripotent state by three approaches. Nature, 2010. 465(7299): p. 704-12.
58. Nicholas, C. R. and A. R. Kriegstein, Regenerative medicine: Cell reprogramming gets direct. Nature, 2010. 463(7284): p. 1031-2.
59. Zhang, J., et al., A human iPSC model of Hutchinson Gilford Progeria reveals vascular smooth muscle and mesenchymal stem cell defects. Cell Stem Cell, 2011. 8(1): p. 31-45.
60. Misteli, T., HGPS-derived iPSCs for the ages. Cell Stem Cell, 2011. 8(1): p.
61. Daley, G. Q., Common themes of dedifferentiation in somatic cell reprogramming and cancer. Cold Spring Harb Symp Quant Biol, 2008. 73: p. 171-4.
62. Gaspar-Maia, A., et al., Open chromatin in pluripotency and reprogramming. Nat Rev Mol Cell Biol, 2011. 12(1): p. 36-47.
63. Daadi, M. M., The common path: tumor suppression in the generation of iPS cells and cancer stem cells. Regen Med, 2010. 5(1): p. 21-2.
64. Gao, P., et al., c-Myc suppression of miR-23a/b enhances mitochondrial glutaminase expression and glutamine metabolism. Nature, 2009. 458(7239): p. 762-5.
65. David, C. J., et al., HnRNP proteins controlled by c-Myc deregulate pyruvate kinase mRNA splicing in cancer. Nature, 2010. 463(7279): p. 364-8.
66. Yuneva, M., et al., Deficiency in glutamine but not glucose induces MYC-dependent apoptosis in human cells. Cell Biol, 2007. 178(1): p. 93-105.
67. Dang, C. V., Rethinking the Warburg effect with Myc micromanaging glutamine metabolism. Cancer Res, 2010. 70(3): p. 859-62.
68. Christofk, H. R., et al., The M2 splice isoform of pyruvate kinase is important for cancer metabolism and tumour growth. Nature, 2008. 452(7184): p. 230-3.
69. Christofk, H. R., et al., Pyruvate kinase M2 is a phosphotyrosine-binding protein. Nature, 2008. 452(7184): p. 181-6.
70. Levine, A. J. and A. M. Puzio-Kuter, The control of the metabolic switch in cancers by oncogenes and tumor suppressor genes. Science, 2010. 330(6009): p. 1340-4.
71. Vander Heiden, M. G., et al., Evidence for an alternative glycolytic pathway in rapidly proliferating cells. Science, 2010. 329(5998): p. 1492-9.
72. West, J. A., et al., A role for Lin28 in primordial germ-cell development and germ-cell malignancy. Nature, 2009. 460(7257): p. 909-13.
73. Viswanathan, S. R., et al., Lin28 promotes transformation and is associated with advanced human malignancies. Nat Genet, 2009. 41(7): p. 843-8.
74. Viswanathan, S. R. and G. Q. Daley, Lin28: A microRNA regulator with a macro role. Cell, 2010. 140(4): p. 445-9.
75. Chang, T. C., et al., Lin-28B transactivation is necessary for Myc-mediated let-7 repression and proliferation. Proc Natl Acad Sci USA, 2009. 106(9): p. 3384-9.
76. Lu, L., et al., Pluripotent factor lin-28 and its homologue lin-28b in epithelial ovarian cancer and their associations with disease outcomes and expression of let-7a and IGF-II. Eur J Cancer, 2009. 45(12): p. 2212-8.
77. Godmann, M., et al., The pluripotency transcription factor Kruppel-like factor 4 is strongly expressed in intratubular germ cell neoplasia unclassified and seminoma. Mol Hum Reprod, 2009. 15(8): p. 479-88.
78. Krizhanovsky, V. and S. W. Lowe, Stem cells: The promises and perils of p53. Nature, 2009. 460(7259): p. 1085-6.
79. Hong, H., et al., Suppression of induced pluripotent stem cell generation by the p53-p21 pathway. Nature, 2009. 460(7259): p. 1132-5.
80. Kawamura, T., et al., Linking the p53 tumour suppressor pathway to somatic cell reprogramming. Nature, 2009. 460(7259): p. 1140-4.
81. Li, H., et al., The Ink4/Arf locus is a barrier for iPS cell reprogramming. Nature, 2009. 460(7259): p. 1136-9.
82. Marion, R. M., et al., A p53-mediated DNA damage response limits reprogramming to ensure iPS cell genomic integrity. Nature, 2009. 460(7259): p. 1149-53.
83. Utikal, J., et al., Immortalization eliminates a roadblock during cellular reprogramming into iPS cells. Nature, 2009. 460(7259): p. 1145-8.
84. Hochedlinger, K. and K. Plath, Epigenetic reprogramming and induced pluripotency. Development, 2009. 136(4): p. 509-23.
85. Yamanaka, S., Ekiden to iPS Cells. Nat. Med, 2009. 15(10): p. 1145-8.
86. Mayshar, Y., et al., Identification and classification of chromosomal aberrations in human induced pluripotent stem cells. Cell Stem Cell, 2010. 7(4): p. 521-31.
87. Ohm, J. E., et al., Cancer-related epigenome changes associated with reprogramming to induced pluripotent stem cells. Cancer Res, 2010. 70(19): p. 7662-73.
88. Duinsbergen, D., et al., Tumors originating from induced pluripotent stem cells and methods for their prevention. Ann NY Acad Sci, 2009. 1176: p. 197-204.
89. Miura, K., et al., Variation in the safety of induced pluripotent stem cell lines. Nat Biotechnol, 2009. 27(8): p. 743-5.
90. Liu, S. V., Are iPS cells really indistinguishable from ES cells? Logical Biology, 2007. 7: p. 66-68.
91. Liu, S. V., Further comments on the nature of iPS cells. Logical Biology, 2007. 7: p. 69-72.
92. Liu, S. V., IPS cells versus ES cells. Logical Biology, 2008. 8(2): p. 47-48.
93. Chin, M. H., et al., Molecular analyses of human induced pluripotent stem cells and embryonic stem cells. Cell Stem Cell, 2010. 7(2): p. 263-9.
94. Chin, M. H., et al., Induced pluripotent stem cells and embryonic stem cells are distinguished by gene expression signatures. Cell Stem Cell, 2009. 5(1): p. 111-23.
95. Newman, A. M. and J. B. Cooper, Lab-specific gene expression signatures in pluripotent stem cells. Cell Stem Cell, 2010. 7(2): p. 258-62.
96. Kim, J., et al., A Myc network accounts for similarities between embryonic stem and cancer cell transcription programs. Cell, 2010. 143(2): p. 313-24.
97. Koche, R. P., et al., Reprogramming factor expression initiates widespread targeted chromatin remodeling. Cell Stem Cell, 2011. 8(1): p. 96-105.
98. Laurent, L. C., et al., Dynamic changes in the copy number of pluripotency and cell proliferation genes in human ESCs and iPSCs during reprogramming and time in culture. Cell Stem Cell, 2011. 8(1): p. 106-18.
99. Salas, A., et al., A critical reassessment of the role of mitochondria in tumorigenesis. PLoS Med, 2005. 2(11): p. e296.
100. Gogvadze, V., S. Orrenius, and B. Zhivotovsky, Mitochondria in cancer cells: what is so special about them? Trends Cell Biol, 2008. 18(4): p. 165-73.
101. Gogvadze, V., S. Orrenius, and B. Zhivotovsky, Mitochondria as targets for cancer chemotherapy. Semin Cancer Biol, 2009. 19(1): p. 57-66.
102. Gogvadze, V., B. Zhivotovsky, and S. Orrenius, The Warburg effect and mitochondrial stability in cancer cells. Mol Aspects Med, 2010. 31(1): p. 60-74.
103. Samudio, I., M. Fiegl, and M. Andreeff, Mitochondrial uncoupling and the Warburg effect: molecular basis for the reprogramming of cancer cell metabolism. Cancer Res, 2009. 69(6): p. 2163-6.
104. Bode, B. P., et al., Molecular and functional analysis of glutamine uptake in human hepatoma and liver-derived cells. Am J Physiol Gastrointest Liver Physiol, 2002. 283(5): p. G1062-73.
105. DeBerardinis, R. J., et al., Beyond aerobic glycolysis: transformed cells can engage in glutamine metabolism that exceeds the requirement for protein and nucleotide synthesis. Proc Natl Acad Sci USA, 2007. 104(49): p. 19345-50.
106. Ganaphthy, V., M. Thangaraju, and P. D. Prasad, Nutrient transporters in cancer: relevance to Warburg hypothesis and beyond. Pharmacol Ther, 2009. 121(1): p. 29-40.
107. Yun, J., et al., Glucose deprivation contributes to the development of KRAS pathway mutations in tumor cells. Science, 2009. 325(5947): p. 1555-9.
108. Tilgner, H. and R. Guigo, From chromatin to splicing: RNA-processing as a total artwork. Epigenetics, 2010. 5(3).
109. Matlin, A. J., F. Clark, and C. W. Smith, Understanding alternative splicing: towards a cellular code. Nat Rev Mol Cell Biol, 2005. 6(5): p. 386-98.
110. Jeyaraj, S., D. M. O'Brien, and D. S. Chandler, MDM2 and MDM4 splicing: an integral part of the cancer spliceome. Front Biosci, 2009. 14: p. 2647-56.
111. Fackenthal, J. D. and L. A. Godley, Aberrant RNA splicing and its functional consequences in cancer cells. Dis Model Mech, 2008. 1(1): p. 37-42.
112. Kim, E., A. Goren, and G. Ast, Insights into the connection between cancer and alternative splicing. Trends Genet, 2008. 24(1): p. 7-10.
113. Malentacchi, F., et al., Alternative splicing variants of carbonic anhydrase IX in human non-small cell lung cancer. Lung Cancer, 2009. 64(3): p. 271-6.
114. Hirschfeld, M., et al., Alternative splicing of Cyr61 is regulated by hypoxia and significantly changed in breast cancer. Cancer Res, 2009. 69(5): p. 2082-90.
115. Biamonti, G. and J. F. Caceres, Cellular stress and RNA splicing. Trends Biochem Sci, 2009. 34(3): p. 146-53.
116. Eilers, M. and R. N. Eisenman, Myc's broad reach. Genes Dev, 2008. 22(20): p. 2755-66.
117. Chang, T. C., et al., Widespread microRNA repression by Myc contributes to tumorigenesis. Nat Genet, 2008. 40(1): p. 43-50.
118. Yoshida, Y., et al., Hypoxia enhances the generation of induced pluripotent stem cells. Cell Stem Cell, 2009. 5(3): p. 237-41.
119. Irimia, D. and M. Toner, Spontaneous migration of cancer cells under conditions of mechanical confinement. Integr Biol (Camb), 2009. 1(8-9): p. 506-12.
120. Feron, O., Pyruvate into lactate and back: from the Warburg effect to symbiotic energy fuel exchange in cancer cells. Radiother Oncol, 2009. 92(3): p. 329-33.
121. Zhou, M., et al., Warburg effect in chemosensitivity: targeting lactate dehydrogenase-A re-sensitizes taxol-resistant cancer cells to taxol. Mol Cancer, 2010. 9: p. 33.
122. Liu, X., et al., Warburg effect revisited: an epigenetic link between glycolysis and gastric carcinogenesis. Oncogene, 2010. 29(3): p. 442-50.

The invention claimed is:

1. A method of screening anti-neoplastic agents by using cells reprogrammed with oncogenic factors (CROFs), comprising the steps of:
determining neoplastic capabilities of CROFs in the presence and in the absence of a test agent;
selecting a test agent which causes a significant reduction in the neoplastic capabilities of the CROFs.

2. The method of claim 1 wherein CROFs are oncogenic cells produced through molecular reprogramming.

3. The method of claim 1 wherein CROFs include incorrectly programmed stem cells.

4. The method of claim 1 wherein the neoplastic capabilities include one or several of the following aspects: elevated aerobic glycolysis, neoplastic anabolism, fast reproduction (proliferation), aggressive invasion or dispersive migration (metastasis).

5. The method of claim 1 wherein the reduction of neoplastic capabilities is judged by comparing a treatment group (in the presence of a single test agent or a combination of testing agents) with a control group (in the absence of such agent(s)) of the same type of CROFs.

6. The method of claim 3 wherein the incorrectly programmed stem cells include induced pluripotent stem cells (iPSCs) which are somatic cells induced with one or more of transcription factors commonly known for generating iPSCs.

7. The method of claim 5 wherein the reduction of neoplastic capabilities can be revealed by examining aerobic glycolysis for an indication includes decreased extracellular acidification rate (ECAR) or decreased expression of glycolytic-specific enzymes.

8. The method of claim 5 wherein the reduction of neoplastic capabilities can be revealed by examining neoplastic anabolism for an indication includes reduced rate of glutaminolysis or reduced expression of glutaminolytic enzymes.

9. The method of claim 5 wherein the reduction of neoplastic capabilities can be revealed by examining cell viability or reproduction rate.

10. The method of claim 5 wherein the reduction of neoplastic capabilities can be revealed by examining cell invasion, migration or metastasis.
